(19) United States
(12) Patent Application Publication
(10) Pub. No.: US 2004/0244074 A1
(43) Pub. Date: Dec. 2, 2004

(54) Identity preserved crop production

(76) Inventors: Keith Jones, Blacksburg, VA (US); Bryan Taliaferro, Center Cross, VA (US)

Correspondence Address: SALIWANCHIK LLOYD & SALIWANCHIK, A PROFESSIONAL ASSOCIATION, PO BOX 142950, GAINESVILLE, FL, 32614-2950, US

(21) Appl. No.: 10/491,768

(22) PCT Filed: Oct. 2, 2002
(86) PCT No.: PCT/US02/31783

Related U.S. Application Data
(60) Provisional application No. 60/326,695, filed on Oct. 2, 2001.

(51) Int. Cl. C12N 15/82; C12N 15/87; A01H 5/00; A01H 5/10

(52) U.S. Cl. 800/278; 800/282; 800/290

(57) ABSTRACT

The subject invention provides methods of selectively recognizing and/or eliminating potentially undesirable soybeans, from the soybean supply and/or selectively preserving specific soybean seed identities comprising: the genetic transformation of small variety soybeans; the separation and isolation of said soybeans; and, optionally, the replanting and harvesting of the soybeans, thereby resulting in stable transgenic lines.

### BACKGROUND OF INVENTION

Agricultural genetic engineering is a rapidly growing industry. Each day scientists are discovering new ways to transfer genetic material into crops so that specific genetic traits may be expressed. Increased crop yield, reduced fertilizer need, production of pharmaceuticals, production of industrial feedstock and intermediates, production of industrial enzymes and pesticidal characteristics are just a few of the many traits that the agricultural industry desires in their crops. On it's face, genetic engineering appears to be largely a beneficial field; however, many of the potential safety issues with regard to the use of genetically engineered crops are recognized and management systems are being put in place.

With much of the focus of genetic engineering aimed towards expressing beneficial genetic traits in crops, much attention has been paid to the potential damaging effects that would result from a commixing of plant products not intended for human consumption with plant products intended for human consumption. The problem of ensuring that such contamination is under study by both the USDA and FDA. The events of 2000 where a non food approved transgenic corn variety was discovered to have been commingled with food use approved corn and to have found its way into over 300 food products show the problems of isolating non-food use plant material. The recall of the non-approved but Starlink contaminated corn which the EPA says poses little or no health risk to consumers is estimated to have cost Aventis over one hundred million dollars.

Soybeans (Glycine max (L.) Merr.) are widely used for human and food animal consumption. Soybean seeds are rich in protein and husbandry techniques are well established. In addition, soybeans have been used for transformation experiments. One soybean variety in particular was extensively used in early transformation experiments. The Peking soybean is a small variety black soybean used because of its relative ease of transformation. (See for example, U.S. Pat. No. 5,569,834). Because of the ease of transformation, soybeans can be used to carry genes for industrial intermediates and pharmaceuticals.

Much like the corn market, the soybean market suffers from the potential for contamination. The introduction of non-food use transgenic soybeans into the food-use soybean supply could in some instances be a problem with serious undesirable consequences. Because of the potential undesirability of transgenic soybeans, any foodstuffs made with the contaminated supply would likely be required to be recalled costing producers millions of dollars and could potentially cause widespread damage to the human population. Therefore, there is a need for a method to recognize and/or selectively eliminate potentially non-desirable soybeans from the soybean supply and selectively recognize and/or preserve specific soybean seed identities.

### BRIEF SUMMARY OF THE INVENTION

The subject invention provides methods of selectively recognizing and/or eliminating potentially undesirable seeds from a seed supply and/or selectively preserving specific seed identities comprising: the genetic transformation of colored seed producing crops; the separation and isolation of transgenic colored seeds from food use colored seeds; and, optionally, the re-planting and harvesting of the transgenic colored seeds, thereby resulting in stable transgenic lines.

Thus, the subject invention, in one embodiment, provides methods of selectively recognizing and/or eliminating potentially undesirable soybeans from the soybean supply and/or selectively preserving specific soybean seed identities comprising: the genetic transformation of small variety soybeans; the separation and isolation of said soybeans; and, optionally, the re-planting and harvesting of the soybeans, thereby resulting in stable transgenic lines.

While the black color of the Peking soybean easily distinguishes it from the food-use commercial soybean seeds that are tan or white in color, its smaller size is also beneficial for two reasons. First, small-seeded soybeans can be identified and separated by screening processes wherein the seeds are filtered through pre-sized screen meshes for separation. Second, while the smaller soybeans yield a lower gram per hundred-count weight, a plant producing such smaller seeds yields two times or more the actual number of seeds. This leads to a reduction in time for producing stable transgenic lines and faster methods of producing genes expressing desired traits.

Seed Coat Coloring Allows Identification and Separation from Other Seeds: Within some genotypes there are cultivars bearing different colored seed coats. For example in soybeans there are two genes determining the color of the seeds. Seeds are either black, brown, yellow or speckled dark on a lighter background depending on the recessive/dominant relationship of the genes. Thus, the use of a cultivar with colored seed coats (black or brown or speckled in the case of soybeans) for a transgenic platform will allow identification of any transgenic seeds within a larger quantity of seeds that may be traded as a commodity.

As another example, corn destined for food use is, typically white, yellow, or a bi-color variety of white and yellow. Other varieties of corn comprise endogenous genes that produce seeds having various colors selected from the group consisting of: blue; red; red, white and blue; plum; maroon; plum; maroon; chinmark; deep purple; pink, rose to dark rose; orange; and various combinations of these colors. Thus, one can create transgenic corn plants by the routine introduction of exogenous genes into these corn plant varieties that give rise to seeds having colors selected from the group consisting of: blue; red; red, white and blue; plum; maroon; plum; maroon; chinmark; deep purple; pink, rose to dark rose; orange; and various combinations of these colors that will allow identification of any transgenic seeds within a larger quantity of seeds that may be traded as a commodity. In other various embodiments of the invention, plants (e.g., soybean or corn plants) may be transformed with exogenous genes that produce a desired color in a seed. Thus, the subject invention aids in reducing the possibility of the introduction of transgenic corn or soybean into the food supply.

Furthermore, differentially colored seeds can be detected by separation equipment using optical scanning devices. Typically in such detection systems, seeds are passed through the field of vision of an optical sensor, such as a camera, that senses the light reflectance of each passing seed. In most such systems insufficient reflectance triggers the operation of an air jet removing the dark or colored seed. In this way a quantity of seeds such as food-use soybeans can be protected from any contaminating transgenic seeds. An example of such optical seed sorter is manufactured by Allen Fruit Co., Inc. of Newburg, Oreg. Model # PSD40490. There are a number of soybean cultivars bearing brown or black seed coats examples in the Virginia Polytechnic Institute and State University collection including, and not limited to, V98-5448, V98-5452 and V96-7198.

One problem with using large-seeded crops such as soybeans for transgenic production of proteins is that after a stable transgenic line is derived, the required characteristics increase by a factor related to the size of the bean, given a reasonable commercial yield, at each generation. Furthermore, the lack of suitable locations with appropriate growing conditions limits the overall crop yield- Small-seeded soybeans, however, compensate for the small size of the seeds by producing more seeds per generation. Small Seeds Allow More Rapid Buildup of a Seed Supply. Utilizing a small seeded cultivar of a plant species in which gene transformation is contemplated will produce a seed supply in less reproductive cycles than for cultivars of normally sized seeds. Such a selection facilitates a more rapid commercialization and production of the cultivar bearing the genetic trait desired.

For example, in two years of testing, the soybean cultivar, V99-2994, has an average seed count of 8330 seeds per pound, while the comparable commercial cultivar, Hutcheson, in the same tests had a seed count of 3179 seeds per pound. In those same two-year tests, average yields of V99-2994 and Hutcheson were 46.1 Bu/Ac and 52.5 Bu/Ac respectively, while plant populations were the same at 160,000 plants per acre. Using these data one may compute the average number of seeds per plant (seed increase ratio), which for V99-2994 was 144, and for Hutcheson was 63. Extra large soybean varieties have, typically, a seed count of about 100 seeds/25 g (or about 1800 seeds per pound).

In spite of a slightly lower yield in terms of bushels per acre, the production by the fifth generation is nearly twenty-four (24) times greater with V99-2994 than with Hutcheson. This may be illustrated by the following formula where reproduction is assumed to begin with one seed. Yields and seed counts are as above.

If: N=number of generations (5)

V=volume in bushels (bulk density of soybeans is 60 pounds per bushel)

R=seed increase ratio

S=number of seeds per pound

Then: V=R^{N}/(60×S)

For Hutcheson at the fifth generation this is 5203 Bu, and for V99-2994 this is 123,884 Bu.

A number of other soybean cultivars such as MFS-553, MFS-591, Camp, V99-2993, V98-3518, V99-3022, and V93-0706 share the characteristic of very small seeds and may be used as an “acceleration platform” for transgenic soybeans.

The transformation of the extra large or small variety soybeans, such as Montague Farms' MFS 553, is performed as known in the art. For example, U.S. Pat. No. 5,569,834 to Hinchee et al. discloses a method for soybean transformation and regeneration. The separation and isolation of the small soybean seeds can be performed by passing the seeds through pre-sized open-mesh screens, or other similar apparatus, allowing only the small transformed soybean varieties to pass through, retaining the large food use soybeans for food- use and eliminating the transformed soybeans from the supply. An example of a screen cleaner that could be used to separated soybeans by size is manufactured by Westrop a/s Slagelse, Denmark. Model (Type) # FP1500XS.

There are also a number of soybean cultivars that have very large seed size that may be separated from other soybeans to recognize or eliminate contamination by very large seeded transgenic soybeans in commodity large seeded soybeans.

The planting and the harvesting of the soybeans are completed as is known in the art. The utilization of the small soybean variety provides a higher seed count per plant, thus reducing the number of generations required to produce stable transgenic soybean lines.

Accordingly, it is an object of the present invention to provide a method of identity preserved soybean crop production. It is a further object of the present invention to provide a method of identity preserved soybean crop production wherein transgenically transformed soybeans are separated and isolated from food-use soybeans. It is a still further object of the present invention to provide a method of identity-preserved soybean crop production wherein transgenically transformed soybean plants produce a higher seed count per plant. It is a still further object of the present invention to provide a method of identity-preserved soybean crop production wherein the time to produce stable transgenic lines of soybeans is greatly reduced. It is a still further object of the present invention to provide a method of identity-preserved soybean crop production wherein the production of transgenically transformed seeds is greatly increased. In various aspects of the subject invention, transgenic soybeans have specific phenotypic traits selected from the group consisting of increased crop yield, reduced fertilizer need, production of pharmaceuticals, production of industrial feedstock and intermediates, production of industrial enzymes, and pesticidal characteristics.

### BRIEF DESCRIPTION OF THE DRAWINGS

FIG. 1 depicts the distribution of seed sizes for small, standard, and large-seeded soybean varieties, as captured by sizing screens.

FIG. 2 is a tabular example of accelerated seed growth increase.

FIG. 3 is a tabular example of seed production increase in bushels.

FIG. 4 is a graphical depiction of seed production increase in bushels.

### DETAILED DISCLOSURE OF THE INVENTION

In order to provide an understanding of a number of terms used in the specification and claims herein, the following definitions are provided.

The term “transformation” as used herein refers to the introduction of exogenous DNA from a foreign cell into a host cell.

The term “stable transgenic line” as used herein refers to the resulting line of plants containing foreign DNA after transformation. As would be apparent to one skilled in the art, the methods taught herein are applicable to mixed soybean samples that contain either small seed or extra large seed transgenic soybeans that are mixed with food-use (non-transgenic) soybeans; alternatively, the methods can be practiced with mixed soybean samples that contain extra large seed and small seed transgenic soybeans in addition to non-transgenic (food use) soybeans. Alternatively, the methods are also applicable to mixed samples of corn that contain non-transgenic and transgenic corn seeds (kernels).

Thus, the subject invention provides a method of identifying seeds, selectively eliminating undesirable seeds, and/or selectively preserving specific seed identities comprising the steps of:

a) optionally, transforming a plant and collecting its seed;

b) providing a mixed sample of seeds comprising:

1. extra-large seed and/or small seed transgenic soybeans and non-transgenic (food-use) soybeans;

2. transgenic colored seed and non-transgenic (food use) seed;

c) separating seed based on size and/or color; and, optionally,

d) replanting the transgenic seed.

In various embodiments of the invention, the soybeans are separated on the basis of size, the basis of color, or on the basis of size and color. Soybeans may be passed through pre-sized open mesh screens and the small seed transgenic soybeans can have a diameter of less than about 6.0 mm (or less than 6.0 mm). In other embodiments, the small seed transgenic soybeans are between about 3.0 mm and 6.0 mm in diameter, preferably, between about 3.5 mm and 6.0 mm in diameter, more preferably between about 4.5 and 5.5 mm in diameter, and even more preferably, about 4.0 mm in diameter. Where extra large seed soybean varieties are used for transformation, soybeans having a diameter of greater than about 7 mm (or at least 7.0 mm) are transgenic and can be, optionally, re-planted. Thus, in various embodiments, transgenic soybeans will have diameters of less than (or less than about) 6.0 mm and/or greater than (or greater than about) 7.0 mm. Non-transgenic soybeans, depending on the embodiments, will have diameters of 6.0 to 6.99 mm in diameter where a soybean sample contains both transgenic extra large and transgenic small seed soybean varieties. In embodiments where a soybean sample contains food use soybeans and only small seed transgenic soybeans, non-transgenic soybeans will have a diameter of about, at least about, or at least 6.0 mm. In embodiments where a soybean sample contains food use soybeans and only extra large seed transgenic soybeans, non-transgenic soybeans will have a diameter of less than about, or less than, 7.0 mm. In various other embodiments of the invention, the method further comprises the replanting of the separated soybeans. The invention may further comprise the harvesting of the replanted soybeans and the production of stable transgenic lines of soybean plants. In any aspect of the subject invention, the transgenic soybeans are transformed with an exogenous gene that encodes a specific trait selected from the group consisting of increased crop yield, reduced fertilizer need, production of pharmaceuticals, production of industrial feedstock and intermediates, production of industrial enzymes, and pesticidal characteristics. Where the seed is separated on the basis of color, non-transgenic (food use) seed has a color selected from the group consisting of: white and yellow bicolor; tan; yellow; and white; and transgenic seed has been transformed with an exogenous gene that produces a desired color or the transgenic seed has a color produced by an endogenous gene and the color is selected from the group consisting of: blue; red; red, white and blue; plum; maroon; plum; maroon; chinmark; deep purple; pink, rose to dark rose; orange; black; brown; yellow; speckled dark on a lighter background; and various combinations of these colors.

In various other embodiments of the invention, the method further comprises the replanting of the separated soybeans. The invention may further comprise the harvesting of the replanted soybeans and the production of stable transgenic lines of soybean plants. In any aspect of the subject invention, the transgenic soybeans are transformed with an exogenous gene that encodes a specific trait selected from the group consisting of increased crop yield, reduced fertilizer need, production of pharmaceuticals, production of industrial feedstock and intermediates, production of industrial enzymes, and pesticidal characteristics.

The subject invention also provides for methods of reducing the number of generations required to produce a stable line of transgenic soybean plants or a method of increasing soybean seed production comprising the steps of:

a) transforming soybeans having small or extra large variety seeds;

b) separating soybeans based on size and/or color; and, optionally,

c) replanting the soybeans.

In various embodiments of the invention, the soybeans are separated on the basis of size, the basis of color, or on the basis of size and color. Soybeans may be passed through pre-sized open mesh screens and the small seed transgenic soybeans can have a diameter of less than about 6.0 mm (or less than 6.0 mm). In other embodiments, the small seed transgenic soybeans are between about 3.0 mm and 6.0 mm in diameter, preferably, between about 3.5 mm and 6.0 mm in diameter, more preferably between about 4.5 and 5.5 mm in diameter, and even more preferably, about 4.0 mm in diameter. Where extra large seed soybean varieties are used for transformation, soybeans having a diameter of greater than about 7 mm (or at least 7.0 mm) are transgenic and can be, optionally, re-planted. Thus, in various embodiments, transgenic soybeans will have diameters of less than (or less than about) 6.0 mm and/or greater than (or greater than about) 7.0 mm. Non-transgenic soybeans, depending on the embodiments, will have diameters of 6.0 to 6.99 mm in diameter where a soybean sample contains both transgenic extra large and transgenic small seed soybean varieties. In embodiments where a soybean sample contains food use soybeans and only small seed transgenic soybeans, non-transgenic soybeans will have a diameter of about, at least about, or at least 6.0 mm. In embodiments where a soybean sample contains food use soybeans and only extra large seed transgenic soybeans, non-transgenic soybeans will have a diameter of less than about, or less than, 7.0 mm. In various other embodiments of the invention, the method further comprises the replanting of the separated soybeans. The invention may further comprise the harvesting of the replanted soybeans and the production of stable transgenic lines of soybean plants. In any aspect of the subject invention, the transgenic soybeans are transformed with an exogenous gene that encodes a specific trait selected from the group consisting of increased crop yield, reduced fertilizer need, production of pharmaceuticals, production of industrial feedstock and intermediates, production of industrial enzymes, and pesticidal characteristics.

In various other embodiments of the invention, the method further comprises the replanting of the separated soybeans. The invention may further comprise the harvesting of the replanted soybeans and the production of stable transgenic lines of soybean plants. In any aspect of the subject invention, the transgenic soybeans are transformed with an exogenous gene that encodes a specific trait selected from the group consisting of increased crop yield, reduced fertilizer need, production of pharmaceuticals, production of industrial feedstock and intermediates, production of industrial enzymes, and pesticidal characteristics.

The subject invention further comprises a methods of reducing the incidence of cross-contamination of food-use (non-transgenic) soybeans with transgenic soybeans comprising:

a) providing a mixed sample of transgenic and food-use (non-transgenic) soybeans

b) separating soybeans based on size and/or color;

wherein separated soybeans having a diameter of about 6.0 to 7.0 mm are suitable for food-use and soybeans having a diameter of less than about (or less than) 6.0 mm or greater than about (or greater than) 7 mm are transgenic and can be, optionally, replanted. Extra large seed soybean varieties used for transformation typically have a diameter of greater than about 7 mm are transgenic and can be, optionally, re-planted.

In various embodiments of the invention, the soybeans are separated on the basis of size, the basis of color, or on the basis of size and color. Soybeans may be passed through pre-sized open mesh screens and the small seed transgenic soybeans can have a diameter of less than about 6.0 mm (or less than 6.0 mm). In other embodiments, the small seed transgenic soybeans are between about 3.0 mm and 6.0 mm in diameter, preferably, between about 3.5 mm and 6.0 mm in diameter, more preferably between about 4.5 and 5.5 mm in diameter, and even more preferably, about 4.0 mm in diameter. Where extra large seed soybean varieties are used for transformation, soybeans having a diameter of greater than about 7 mm (or at least 7.0 mm) are transgenic and can be, optionally, re-planted. Thus, in various embodiments, transgenic soybeans will have diameters of less than (or less than about) 6.0 mm and/or greater than (or greater than about) 7.0 mm. Non-transgenic soybeans, depending on the embodiments, will have diameters of 6.0 to 6.99 mm in diameter where a soybean sample contains both transgenic extra large and transgenic small seed soybean varieties. In embodiments where a soybean sample contains food use soybeans and only small seed transgenic soybeans, non-transgenic soybeans will have a diameter of about, at least about, or at least 6.0 mm. In embodiments where a soybean sample contains food use soybeans and only extra large seed transgenic soybeans, non-transgenic soybeans will have a diameter of less than about, or less than, 7.0 mm. In various other embodiments of the invention, the method further comprises the replanting of the separated soybeans. The invention may further comprise the harvesting of the replanted soybeans and the production of stable transgenic lines of soybean plants. In any aspect of the subject invention, the transgenic soybeans are transformed with an exogenous gene that encodes a specific trait selected from the group consisting of increased crop yield, reduced fertilizer need, production of pharmaceuticals, production of industrial feedstock and intermediates, production of industrial enzymes, and pesticidal characteristics.

The subject invention also provides a method of selectively recognizing and/or eliminating potentially undesirable corn (Zea maize L.) germ lines from the corn supply and/or selectively preserving specific corn seed identities by color. There are well known cultivars of corn that have are strongly blue, red or brightly variegated. Examples include but are not limited to the Yubraska family of hybrids offered by Clarkeson Grain, 320 East South St. Serro Gordo, Ill. 61818, include the brightly colored Más Maíz™ Deep Indigo Blue and Más Maíz™ Red Flint varieties and multicolored Indian Corn offered by Southern States, Southern States Cooperative, Inc. 6606 West Broad St. Richmond, Va. 23230-1717. Corn is an open pollinated crop and its pollen can travel up to one kilometer. Pollination by a colored corn cultivar carrying a dominant color gene will result in colored kernels developing in food use yellow or white corn and, thus, allows cross pollination to be detected and preventing or reducing the occurrence of introduction of transformed corn into the food supply. Currently existing transgenes in corn can be transferred to colored corn by traditional breeding techniques.

The following example illustrates the procedures for practicing the invention. This example should not be construed as limiting.

### EXAMPLE 1

Small variety soybean sizes can be used in the practice of this invention. The seed size distribution for a typical small soybean variety is from less than 3 mm in diameter to about 6 mm in diameter. With the greatest percentage of measured sample at about 4 mm in diameter, the percentage of measured sample rapidly declines as the seed size approaches 6 mm in diameter.

A small soybean variety, such as Montague Farms' MFS-555, is selected for its characteristic size and ease of transformation. The soybean is transformed to carry a gene to express a specific trait. For example, the soybean may be transformed to express a pesticidal feature, carry an industrial intermediate or carry a pharmaceutical. Transformed small variety soybean seeds are planted and allowed to grow until ready for harvest. When the seeds are ready for harvest, they are picked and separated according to size.

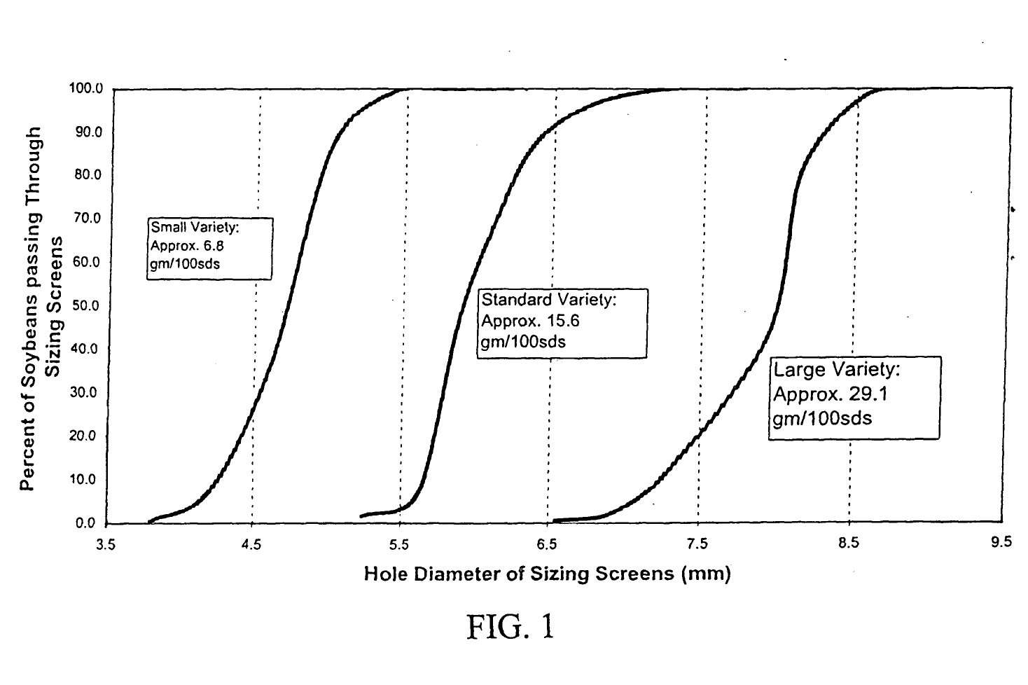

By selecting only small variety soybean seeds for transformation, the ability for separating the potentially toxic seeds from the overall soybean seed supply is markedly increased. FIG. 1 illustrates the percentage of seed varieties passing through pre-sized open mesh screens and the approximate weight (in grams) per hundred seeds. While small soybean seeds range in size from about 3.5 mm in diameter to about 6.0 mm in diameter, standard soybean seeds range from about 5.5 mm to about 7.0 mm in diameter and large soybean seeds range from about 6.5 mm to about 9.0 mm in diameter. As illustrated by the chart, each variety of soybeans may be isolated from another variety by filtration through sizing screens. Also shown in the chart is a slight overlap in seed variety distribution at certain mesh (hole) diameter sizes. A slight adjustment in the hole diameter size can essentially eliminate the ability of cross-contamination of transformed small variety seeds with non-transformed food-use soybean seeds.

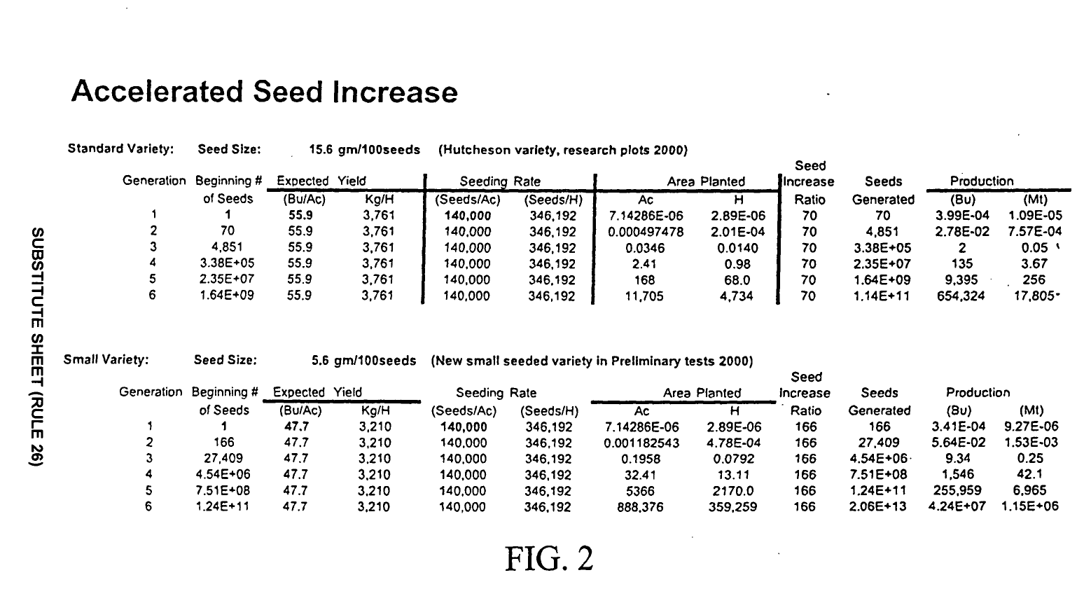

Once the transformed small variety soybean is isolated, it is utilized for re-planting, transformation, or intended use. Soybean seeds that are re-planted become soybean plants producing small variety soybeans expressing the desired genetic traits. In addition, small variety soybean plants produce two or more times the number of soybean seeds per plant than the standard soybean variety plants. As shown in FIG. 2, the small variety soybean plants produce an accelerated seed increase over the standard variety soybean plant. This accelerated seed growth reduces the number of generations needed to produce a stable transgenic line of soybean plants.

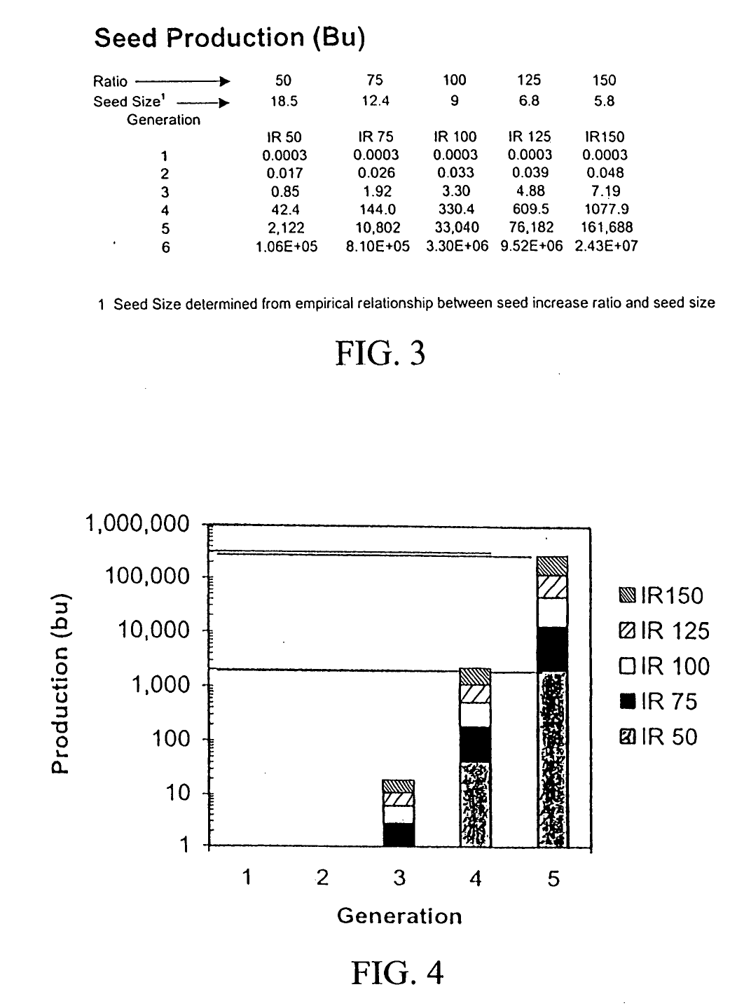

Another benefit of utilizing small variety soybean seeds is the overall seed production in bushels. While the seeds resulting from the small variety soybean are smaller than the standard variety soybean, the higher yield in raw numbers of seeds creates an overall increase in production. FIGS. 3 and 4 illustrate the correlation between the seed size variety and overall production in bushels of seeds.

By selecting and transforming only small variety soybeans, the possibility of cross-contamination with food-use soybean supplies can be greatly reduced by employing the method disclosed and claimed herein. Small variety soybeans can also be filtered from the food-use supply through pre-sized open mesh screens and re-planted to produce a soybean plant producing two or more times as many soybean seeds as the standard variety soybean plant. This yields larger raw numbers of soybeans resulting in increased overall seed production. Furthermore, a stable transgenic line is created, thus preserving the identity of the soybean crop.

All patents, patent applications, provisional applications, and publications referred to or cited herein are incorporated by reference in their entirety, including all figures and tables, to the extent they are not inconsistent with the explicit teachings of this specification.

It should be understood that the example and embodiment described herein is for illustrative purposes only and that various modifications or changes in light thereof will be suggested to persons skilled in the art and are to be included within the spirit and purview of this application and the scope of the appended claims.

1-61. (Canceled)

62. A method of identifying seeds, selectively eliminating undesirable seeds, and/or selectively preserving specific seed identities comprising the steps of:
a) optionally, transforming a plant and collecting its seed;
b) providing a mixed sample of seeds comprising:
1. extra-large seed, small seed, or combinations of extra-large and small seed transgenic soybeans and non-transgenic (food-use) soybeans;
2. transgenic colored seed and non-transgenic (food use) seed;
c) separating seed based on size, color or both size and color; and, optionally,
d) replanting the transgenic seed.

63. The method according to claim 62, wherein the seed is separated on the basis of color and non-transgenic (food use) seed has a color selected from the group consisting of: white and yellow bicolor; tan; yellow; and white;
and transgenic seed has been transformed with an exogenous gene that produces a desired color or the transgenic seed has a color produced by an endogenous gene and the color is selected from the group consisting of: blue; red; red, white and blue; plum; maroon; plum; maroon; chimnark; deep purple; pink, rose to dark rose; orange; black; brown; yellow; speckled dark on a lighter background; and various combinations of these colors.

64. A method of reducing the number of generations required to produce a stable line of transgenic soybean plants or a method of increasing soybean seed production comprising the steps of:
a) transforming small seed or extra large seed variety soybeans;
b) separating transformed soybeans based on: a) size, b) color, or c) both size and color; and, optionally,
c) replanting the transformed soybeans.

65. The method according to claim 64, wherein the soybeans are separated on the basis of size.

66. The method according to claim 64, wherein the soybeans are separated on the basis of color.

67. The method according to claim 64, wherein the soybeans are separated on the basis of size and color.

68. The method according to claim 65, wherein the soybeans are passed through pre-sized open mesh screens.

69. The method according to claim 64, wherein the soybeans are between about 3.0 mm and 6.0 mm in diameter.

70. The method according to claim 64, wherein the soybeans are between about 3.5 mm and 6.0 mm in diameter.

71. The method according to claim 64, wherein the soybeans are between about 4.5 and 5.5 mm in diameter.

72. The method according to claim 64, wherein the soybeans are about 4.0 mm in diameter.

73. The method according to claim 64, wherein the method further comprises the replanting of the separated transgenic soybeans.

74. The method according to claim 73, further comprising the harvesting of the replanted soybeans.

75. The method according to claim 74, wherein stable transgenic lines of soybean plants are produced.

76. The method according to claim 64, wherein the soybeans are transformed with an exogenous gene that encodes a specific trait.

77. The method according to claim 76, wherein the specific trait is selected from the group consisting of: a) pesticidal activity; b) a pharmaceutical; c) an industrial intermediate; d) a feedstock; e) increased crop yield; f) reduced fertilizer need; and g) production of industrial enzymes.

78. A method of reducing the incidence of cross-contamination of food-use (non-transgenic) soybeans with transgenic soybeans comprising:
a) providing a mixed sample of transgenic and food-use (non-transgenic) soybeans
b) separating soybeans based on: a) size, b) color or c) both size and color;
wherein separated soybeans having a diameter of about 6.0 mm or greater are suitable for food-use and soybeans having a diameter of less than about 6.0 mm are transgenic and can be, optionally, replanted.

79. The method according to claim 78, wherein the soybeans are separated on the basis of size.

80. The method according to claim 78, wherein the soybeans are separated on the basis of color.

81. The method according to claim 78, wherein the soybeans are separated on the basis of size and color.
